(19) United States
(12) Patent Application Publication
(10) Pub. No.: US 2008/0300642 A1
(43) Pub. Date: Dec. 4, 2008

(54) REGENERATION TREATMENT APPARATUS, OPERATING METHOD THEREOF, AND REGENERATION TREATMENT METHOD

(75) Inventors: Masashi INAGAKI, Yotsukaido-shi (JP); Masaru SUGIMACHI, Osaka (JP); Meihua LI, Osaka (JP); Can ZHENG, Osaka (JP); Masatoshi KOBAYASHI, Tokyo (JP); Kazuo SHIMIZU, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignees: JAPAN AS REPRESENTED BY PRESIDENT OF NATIONAL CARDIOVASCULAR CENTER, Osaka (JP); OLYMPUS CORPORATION, Tokyo (JP)

(21) Appl. No.: 12/129,360

(22) Filed: May 29, 2008

Related U.S. Application Data
(60) Provisional application No. 60/932,282, filed on May 30, 2007.

(30) Foreign Application Priority Data
May 20, 2008 (JP) 2008-132361

(51) Int. Cl. A61N 1/362

(52) U.S. Cl. 607/14

(57) ABSTRACT

The cell viability in cell transplantation is improved so as to achieve sufficient repair of organ. There is provided a regeneration treatment apparatus comprising: a heart rate detector which detects a heart rate of a patient; a memory part which stores a heart rate prior to stimulation; stimulating electrodes which stimulate a vagus nerve that controls an organ having transplanted cells; and a control unit which controls an intensity of a stimulation signal to be output from the stimulating electrodes to the vagus nerve so that the heart rate of the patient detected by the heart rate detector is decreased by 5 to 20% as compared to the state prior to the stimulation.

### CROSS-REFERENCE TO RELATED APPLICATIONS

This application claims the benefit of U.S. Provisional Application No. 60/932,282, filed May 30, 2007.

### BACKGROUND OF THE INVENTION

1. Field of the Invention

The present invention relates to a regeneration treatment apparatus, an operating method thereof, and a regeneration treatment method.

This application is based on Japanese Patent Application No. 2008-132361, the content of which is incorporated herein by reference.

2. Description of Related Art

Conventionally, tissue engineering for heart failure treatment have employed techniques such as transplantation of transgenic cells and transplantation of other cells (refer to PCT International Publication No. WO 2001/048151 Pamphlet, PCT International Publication No. WO 2004/045666 Pamphlet, and PCT International Publication No. WO 2003/059375 Pamphlet).

However, such regenerative treatments through cell transplantation have concerns in that the cell viability of transplanted cells is poor and thus repair of organ is insufficient even if stem cells are transplanted.

### BRIEF SUMMARY OF THE INVENTION

The present invention takes the above situation into consideration with an object of providing a regeneration treatment apparatus, an operating method thereof, and a regeneration treatment method, capable of improving the cell viability in cell transplantation so as to achieve sufficient repair of organ.

In order to achieve the above object, the present invention provides the following solutions.

The present invention provides a regeneration treatment apparatus comprising: a heart rate detector which detects a heart rate of a patient; a memory part which stores a heart rate prior to stimulation; stimulating electrodes which stimulate a vagus nerve that controls an organ having transplanted cells; and a control unit which controls an intensity of a stimulation signal to be output from the stimulating electrodes to the vagus nerve so that the heart rate of the patient detected by the heart rate detector is decreased by 5 to 20% as compared to the state prior to the stimulation.

According to the present invention, stimulation to the vagus nerve from the stimulating electrodes can increase engraftment of transplanted cells. In this case, the heart rate of the patient detected by the heart rate detector is previously stored in the memory part prior to stimulation, and the control unit controls the intensity of the stimulation signal so that the heart rate detected after the stimulation is decreased by 5 to 20% as compared to the heart rate prior to the stimulation that has been stored in the memory part, by which repair of the organ having transplanted cells can be enhanced.

In the above invention, the stimulation signal may take a pulse-like form having a frequency of about 10 Hz, a stimulation voltage of about 0.1 to 7.5 V, and a pulse width of about 0.4 to 3 msec.

Moreover, in the above invention, the stimulation signal may be either intermittently or continuously output from the stimulating electrodes.

Furthermore, in the above invention, the stimulation signal may be intermittently output from the stimulating electrodes by repetition of one-minute stimulation pattern consisting of: a continuous stimulation period for 5 to 30 seconds from the stimulating electrodes; and a nonstimulation period for the rest of time.

Moreover, in the above invention, the stimulation signal may be an electrical stimulation signal consisting of biphasic pulses. In the case of electrical stimulation, application of monophasic pulses makes a tissue charged either positively or negatively. Therefore, application of an electrical stimulation signal consisting of biphasic pulses can keep the tissue from being charged.

The present invention also provides an operating method of a regeneration treatment apparatus, comprising: operating a heart rate detector which detects a heart rate of a patient; operating a memory part which stores a heart rate prior to stimulation; operating a stimulation signal generator which generates a stimulation signal to stimulate a vagus nerve; operating a determination unit which determines whether or not the heart rate detected by the heart rate detector is decreased by 5 to 20% as compared to the heart rate stored in the memory part; and operating a control unit which increases the intensity of the stimulation signal generated from the stimulation signal generator if the determination unit determines that the reduction of the heart rate is less than 5%, and lowers the intensity of the stimulation signal if the determination unit determines that the reduction of the heart rate exceeds 20%.

In the above invention, the stimulation signal may take a pulse-like form having a frequency of about 10 Hz, a stimulation voltage of about 0.1 to 7.5 V, and a pulse width of about 0.4 to 3 msec.

Moreover, in the above invention, the stimulation signal may be either intermittent or continuous.

Furthermore, in the above invention, the stimulation signal may be an intermittent stimulation signal achieved by repetition of one-minute stimulation pattern consisting of: a continuous stimulation period for 5 to 30 seconds; and a nonstimulation period for the rest of time.

Moreover, in the above invention, the stimulation signal may be an electrical stimulation signal consisting of biphasic pulses.

The present invention also provides a regeneration treatment method, comprising: transplanting cells into an organ; and stimulating a vagus nerve that controls the organ having the transplanted cells.

In the above invention, the stimulation site of the vagus nerve is preferably a vagus nerve ganglion or a vagus nerve branch. Stimulation to such a stimulation site enables selective stimulation to an organ having transplanted cells without stimulating other organs, and enables prevention of the occurrence of side effects in other organs.

Moreover, the above invention may also be such that the heart rate of the patient is detected and the intensity of stimulation to the vagus nerve is adjusted so that the detected heart rate is set to a predetermined heart rate.

In this case, the stimulation intensity is preferably adjusted so that the detected heart rate is decreased by 5 to 20% as compared to the state prior to the stimulation.

In the above invention, the stimulation to the vagus nerve is preferably a pulse-like stimulation having a frequency of about 10 Hz, a stimulation voltage of about 0.1 to 7.5 V, and a pulse width of about 0.4 to 3 msec.

Moreover, in the above invention, the pulse-like stimulation may be either intermittently or continuously performed.

Furthermore, in the above invention, the pulse-like stimulation may be intermittently performed by repetition of one-minute stimulation pattern consisting of: a continuous stimulation period for 5 to 30 seconds; and a nonstimulation period for the rest of time.

Moreover, in the above invention, the pulse-like stimulation is preferably an electrical stimulation performed by biphasic pulses.

In the above invention, a positive electrode and a negative electrode for stimulation are preferably spaced 2 to 5 mm apart to be arranged on the vagus nerve. If these electrodes are spaced too close, the area to be stimulated becomes too narrow. If these electrodes are spaced too apart, the efficiency of stimulation is lowered, requiring a large amount of energy. Such an arrangement with a 2- to 5-mm space enables stimulation without such a concern.

In the above invention, the negative electrode is preferably arranged closer to the organ than the positive electrode.

By so doing, excitation to the vagus nerve generated by the negative electrode can be readily transmitted to the organ side, and acetylcholine released from vagus nerve endings improves the viability of transplanted cells.

The present invention exerts an effect capable of improving the cell viability in cell transplantation so as to achieve sufficient repair of organ.

### BRIEF DESCRIPTION OF THE SEVERAL VIEWS OF THE DRAWINGS

FIG. 1 is a schematic diagram showing a regeneration treatment apparatus according to one embodiment of the present invention.

FIG. 2 is a block diagram showing a control unit of the regeneration treatment apparatus of FIG. 1.

FIG. 3 is a flowchart showing a regeneration treatment method using the regeneration treatment apparatus of FIG. 1.

FIG. 4 is a flowchart showing the regeneration treatment method, continued from FIG. 3.

FIG. 5 is a flowchart showing the regeneration treatment method, continued from FIG. 3.

FIG. 6 is a flowchart showing the regeneration treatment method, continued from FIG. 3.

FIG. 7 is a schematic diagram showing an example of arrangement of stimulating electrodes in the regeneration treatment method of FIG. 3.

FIG. 8 is a graph showing time series changes in the left ventricular diastolic dimension of rat hearts, in one example of the regeneration treatment method of FIG. 3.

FIG. 9 is a graph showing time series changes in the left ventricular systolic dimension of rat hearts, in the same example as in FIG. 8.

FIG. 10 is a graph showing time series changes in the left ventricular fractional shortening of rat hearts, in the same example as in FIG. 8.

FIG. 11 is a graph showing time series changes in the left ventricular ejection fraction of rat hearts, in the same example as in FIG. 8.

FIG. 12 is a graph showing the end-systolic elastance of left ventricle of rat hearts, in the same example as in FIG. 8.

FIG. 13 is a graph showing the left ventricular end-diastolic pressure of rat hearts, in the same example as in FIG. 8.

FIG. 14 is a graph showing the cardiac index of rat hearts, in the same example as in FIG. 8.

FIG. 15 is a graph showing the maximum negative first derivative of left ventricular pressure of rat hearts, in the same example as in FIG. 8.

FIG. 16 is a graph showing the ventricular weight normalized by body weight of rat hearts, in the same example as in FIG. 8.

FIG. 17 is a graph showing number of viable transplanted cells of rat hearts, in the same example as in FIG. 8.

FIG. 18 is a graph showing the microvessel density of rat hearts, in the same example as in FIG. 8.

### DETAILED DESCRIPTION OF THE INVENTION

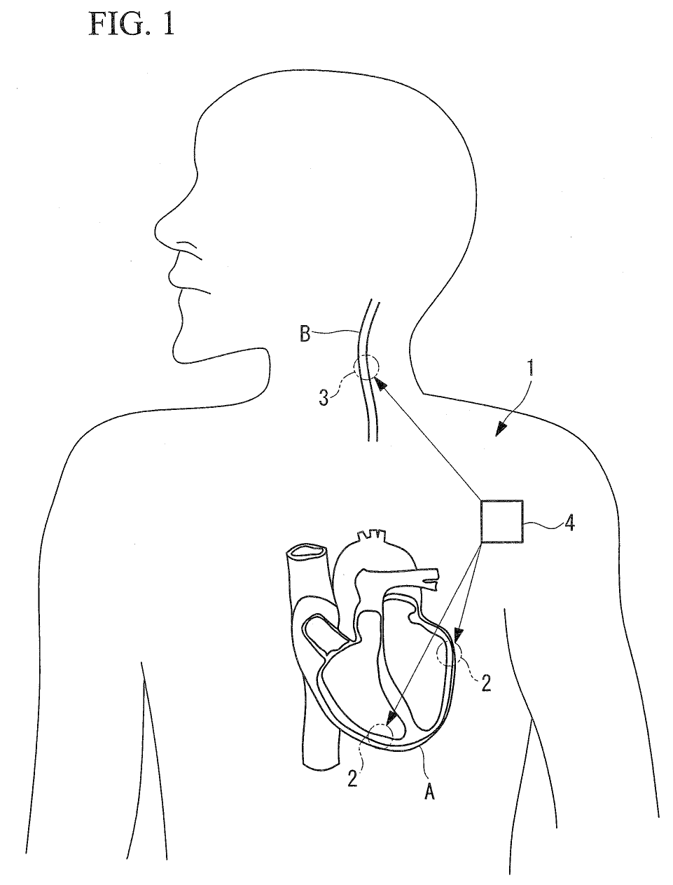

Hereunder is a description of a regeneration treatment apparatus 1, an operating method thereof, and a regeneration treatment method according to one embodiment of the present invention, with reference to FIG. 1 to FIG. 18.

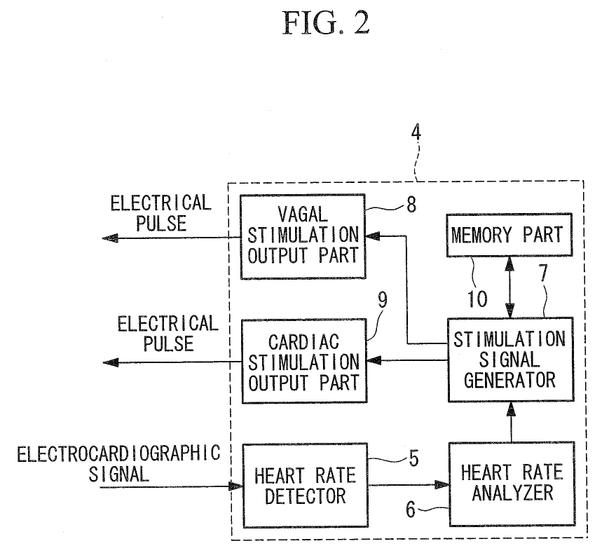

As shown in FIG. 1 and FIG. 2, the regeneration treatment apparatus 1 according to the present embodiment comprises a bioelectricity electrode 2 attached to the endocardial or epicardial surface of the heart A, stimulating electrodes 3 arranged on the vagus nerve B, and a control unit 4 which controls stimulation to the heart A through the bioelectricity electrode 2 and/or stimulation to the vagus nerve B through the stimulating electrodes 3, based on an electrocardiographic signal detected by the bioelectricity electrode 2.

As shown in FIG. 2, the control unit 4 comprises: a heartbeat detector 5 which detects heartbeats from the electrocardiographic signal that has been detected by the bioelectricity electrode 2; a heart rate analyzer 6 which analyzes the state of the heart A, based on the heartbeats detected by the heartbeat detector 5; a stimulation signal generator 7 which generates a stimulation signal in accordance with the analysis result by the heart rate analyzer 6; a vagal stimulation output part 8 which outputs the stimulation signal generated by the stimulation signal generator 7, to the stimulating electrodes 3; and a cardiac stimulation output part 9 which outputs the stimulation signal generated by the stimulation signal generator 7, to the bioelectricity electrode 2.

The heart rate analyzer 6 determines whether the heart A is in a healthy state or in an abnormal state (bradycardia, ventricular fibrillation, or ventricular tachycardia), from the time-series information on the heartbeat of the heart A sent from the heartbeat detector 5, and sends various outputs on the basis of the determination result.

Specifically, if the heart A is in an abnormal state, the heart rate analyzer 6 diagnoses the type of state of the heart A is in; bradycardia, ventricular fibrillation, or ventricular tachycardia, from the time-series information on the heartbeat.

According to the diagnosis result, the stimulation signal generator 7 generates a stimulation signal to be output to the bioelectricity electrode 2 and/or the stimulating electrodes 3.

Specifically, if the heart rate analyzer 6 determines that the state of the heart A is ventricular tachycardia, the stimulation signal generator 7 makes settings of electrical pulses to be output to the stimulating electrodes 3 through the vagal stimulation output part 8 (described later), and outputs the electrical pulses to the vagal stimulation output part 8. Simultaneously, the stimulation signal generator 7 also makes settings of electrical pulses at a rhythm rapider than the heart rate of the ventricular tachycardia, or a high energy shock, and outputs the electrical pulses or high energy shock to the bioelectricity electrode 2 through the cardiac stimulation output part 9.

Moreover, if the heart rate analyzer 6 determines that the state of the heart A is bradycardia, the stimulation signal generator 7 makes settings of electrical pulses at a rhythm of about the same degree as the normal heartbeat for the heart A, and outputs the electrical pulses to the bioelectricity electrode 2 through the cardiac stimulation output part 9. Moreover, if it is determined that the state of the heart A is ventricular fibrillation, the stimulation signal generator 7 generates defibrillation pulses for applying a high energy electric shock to all cardiac cells, each of which is disorderly contracting, through the bioelectricity electrode 2.

Furthermore, the stimulation signal generator 7 is connected to a memory part 10 which stores the heart rate prior to the stimulation to the vagus nerve B. If the heart rate analyzer 6 determines that the state of the heart A is normal, the stimulation signal generator 7 generates a stimulation signal based on the heart rate stored in the memory part 10.

Specifically, the stimulation signal generator 7 determines the intensity of the stimulation signal according to whether or not the heart rate detected after the stimulation to the vagus nerve B is decreased by 5 to 20% as compared to the heart rate prior to the stimulation that has been stored in the memory part 10. The stimulation signal generator 7 is designed to increase the intensity of the stimulation signal if the reduction rate is less than 5%, and to lower the intensity of the stimulation signal if the heart rate is decreased by 20% or more.

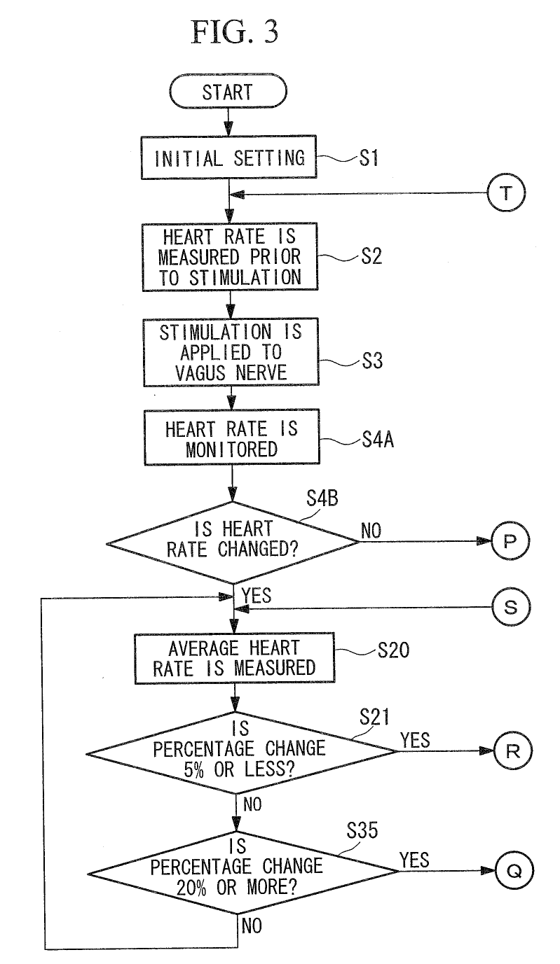

More specifically, the regeneration treatment apparatus 1 according to the present embodiment is operated in accordance with the flowchart shown in FIG. 3.

First, initial values of stimulation conditions are set (Step S1). The heart rate is measured for a predetermined time prior to stimulation and stored in the memory part 10 (Step S2). Next, stimulation is applied to the vagus nerve B at a predetermined stimulation intensity for a predetermined time (Step S3), and changes in the heart rate are monitored (Steps S4A and S4B).

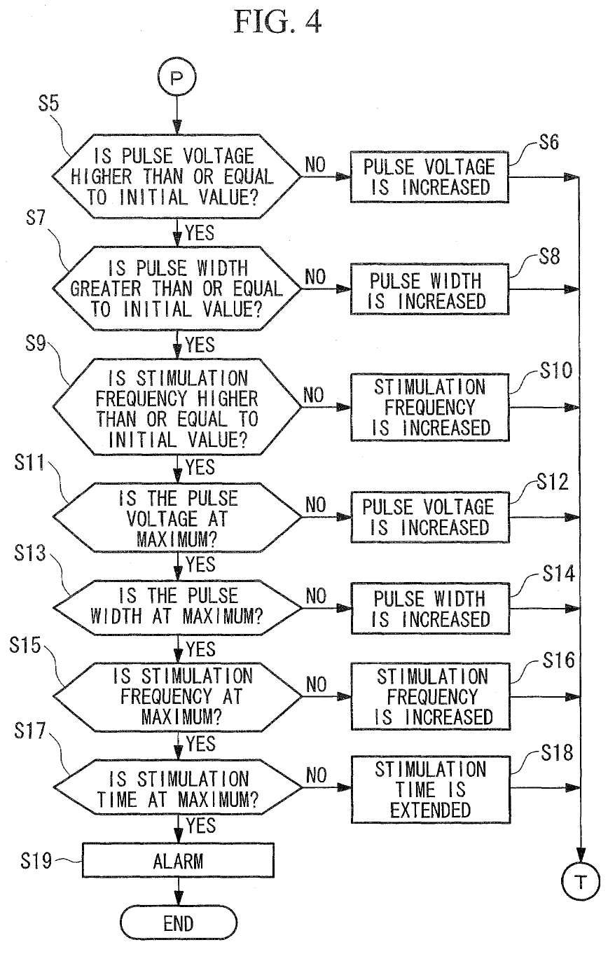

If the heart rate remains unchanged; as FIG. 4, it is determined whether or not the pulse voltage is higher than or equal to the initial value (Step S5). If the pulse voltage is lower than the initial value, the voltage of electrical pulses is increased at a predetermined increment (Step S6), and the steps from Step S2 are repeated. If the pulse voltage is higher than or equal to the initial value, it is determined whether or not the pulse width is greater than or equal to the initial value (Step S7). As a result of the determination, if the pulse width is smaller than the initial value, the pulse width of electrical pulses is increased at a predetermined increment (Step S8), and the steps from Step S2 are repeated.

Moreover, if the pulse width is greater than or equal to the initial value, it is determined whether or not the stimulation frequency is higher than or equal to the initial value (Step S9). As a result of the determination, if the stimulation frequency is lower than initial value, the stimulation frequency of electrical pulses is increased at a predetermined increment (Step S10), and the steps from Step S2 are repeated.

If the stimulation frequency is higher than or equal to the initial value, it is determined whether or not the pulse voltage is at its maximum (Step S11). As a result of the determination, if the pulse voltage is not at its maximum, the voltage of electrical pulses is increased at a predetermined increment (Step S12), and the steps from Step S2 are repeated.

If the pulse voltage is at its maximum, it is determined whether or not the pulse width is at its maximum (Step S13). As a result of the determination, if the pulse width is not at its maximum, the pulse width of electrical pulses is increased at a predetermined increment (Step S14), and the steps from Step S2 are repeated.

If the pulse width is at its maximum, it is determined whether or not the stimulation frequency is at its maximum (Step S15). As a result of the determination, if the stimulation frequency is not at its maximum, the stimulation frequency of electrical pulses is increased at a predetermined increment (Step S16), and the steps from Step S2 are repeated.

If the stimulation frequency is at its maximum, it is determined whether or not the stimulation time is at its maximum (Step S17). As a result of the determination, if the stimulation time is not at its maximum, the stimulation time is extended at a predetermined increment (Step S18), and the steps from Step S2 are repeated.

Moreover, if the stimulation time is at its maximum, an alarm is activated (Step S19) to stop the process.

As a result of the stimulation to the vagus nerve B, if the heart rate is changed; for example, an average heart rate for 1 to 5 minutes is measured within the stimulation/nonstimulation period (Step S20), and the measured average heart rate is compared to the heart rate prior to the stimulation that has been stored in the memory part 10.

As a result of the comparison, it is determined whether or not the average heart rate is changed by 5% or less as compared to the heart rate prior to the stimulation (Step S21).

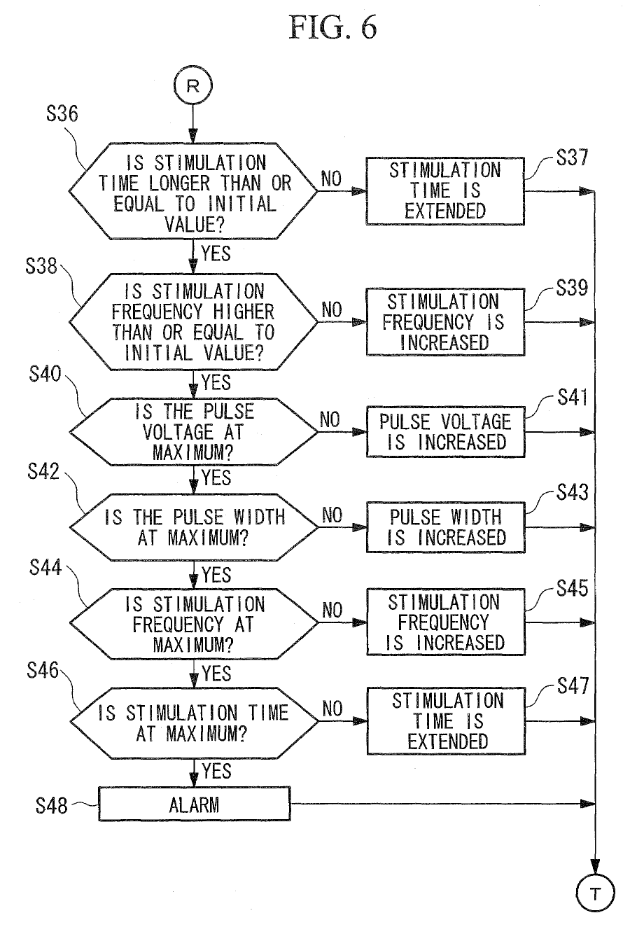

As a result of the determination, if the percentage change is 5% or less, as shown in FIG. 6, it is determined whether or not the stimulation time is longer than or equal to the initial value (Step S36). As a result of the determination, if the stimulation time is shorter than the initial value, the stimulation time is extended by a predetermined time (Step S37), and the steps from Step S2 are repeated.

If the stimulation time is longer than or equal to the initial value, it is determined whether or not the stimulation frequency is higher than or equal to the initial value (Step S38). As a result of the determination, if the stimulation frequency is lower than the initial value, the stimulation frequency of electrical pulses is increased by a predetermined frequency (Step S39), and the steps from Step S2 are repeated.

If the stimulation frequency is higher than or equal to the initial value, it is determined whether or not the pulse voltage is at its maximum (Step S40). As a result of the determination, if the pulse voltage is not at its maximum, the pulse voltage of electrical pulses is increased (Step S41), and the steps from Step S2 are repeated.

If the pulse voltage is at its maximum, it is determined whether or not the pulse width is at its maximum (Step S42). As a result of the determination, if the pulse width is not at its maximum, the pulse width of electrical pulses is increased (Step S43), and the steps from Step S2 are repeated.

If the pulse width is at its maximum, it is determined whether or not the stimulation frequency is at its maximum (Step S44). As a result of the determination, if the stimulation frequency is not at its maximum, the stimulation frequency of electrical pulses is increased by a predetermined frequency (Step S45), and the steps from Step S2 are repeated.

If the stimulation frequency is at its maximum, it is determined whether or not the stimulation time is at its maximum (Step S46). As a result of the determination, if the stimulation time is not at its maximum, the stimulation time of electrical pulses is extended by a predetermined time (Step S47), and the steps from Step S2 are repeated.

If the stimulation time is at its maximum, an alarm is activated (Step S48) to repeat the steps from Step S2.

Moreover, as a result of the comparison between the measured average heart rate and the heart rate prior to the stimulation that has been stored in the memory part 10, it is determined whether or not the average heart rate is changed by 5% or more as compared to the heart rate prior to the stimulation (Step S21).

As a result of the determination, if the percentage change is 5% or more, it is determined whether or not the average heart rate is changed by 20% or more as compared to the heart rate prior to the stimulation (Step S35).

As a result of the determination, if the percentage change is less than 20%, the steps from Step S20 are repeated.

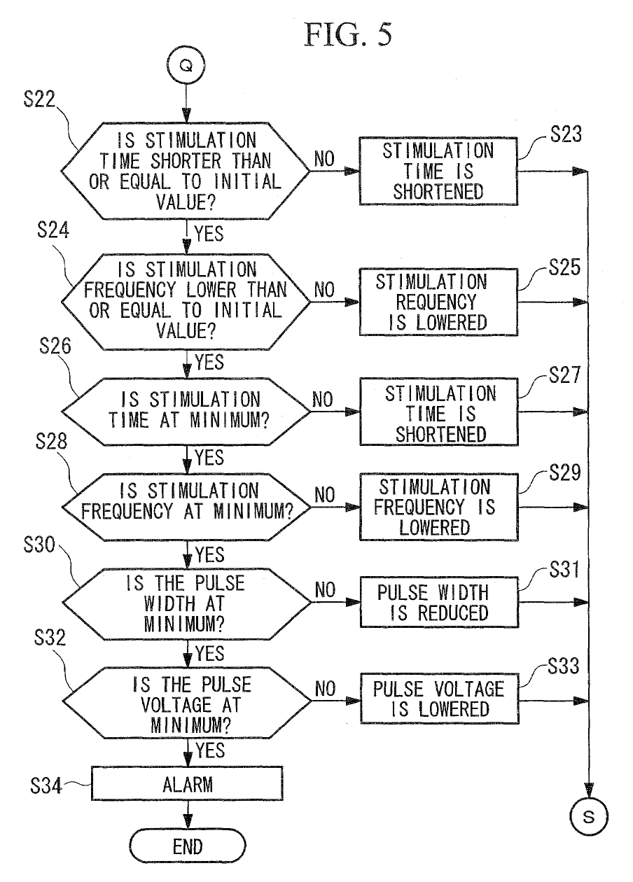

If the percentage change is 20% or more, as shown in FIG. 5, it is determined whether or not the stimulation time is shorter than or equal to the initial value (Step S22). As a result of the determination, if the stimulation time is longer than the initial value, the stimulation time is shortened by a predetermined time (Step S23), and the steps from Step S20 are repeated.

If the stimulation time is shorter than or equal to the initial value, it is determined whether or not the stimulation frequency is lower than or equal to the initial value (Step S24). As a result of the determination, if the stimulation frequency is higher than the initial value, the stimulation frequency of electrical pulses is lowered by a predetermined frequency (Step S25), and the steps from Step S20 are repeated.

If the stimulation frequency is lower than or equal to the initial value, it is determined whether or not the stimulation time is at its minimum (Step S26). As a result of the determination, if the stimulation time is not at its minimum, the stimulation time is shortened by a predetermined time (Step S27), and the steps from Step S20 are repeated.

If the stimulation time is at its minimum, it is determined whether or not the stimulation frequency is at its minimum (Step S28). As a result of the determination, if the stimulation frequency is not at its minimum, the stimulation frequency of electrical pulses is lowered by a predetermined frequency (Step S29), and the steps from Step S20 are repeated.

If the stimulation frequency is at its minimum, it is determined whether or not the pulse width is at its minimum (Step S30). As a result of the determination, if the pulse width is not at its minimum, the pulse width of electrical pulses is reduced (Step S31), and the steps from Step S20 are repeated.

If the pulse width is at its minimum, it is determined whether or not the pulse voltage is at its minimum (Step S32). As a result of the determination, if the pulse width is not at its minimum, the pulse voltage of electrical pulses is lowered (Step S33), and the steps from Step S20 are repeated.

Moreover, if the pulse voltage is at its minimum, an alarm is activated (Step S34) to stop the process.

Electrical pulses to be output from the stimulation signal generator 7 through the vagal stimulation output part 8 are set to have a frequency of about 10 Hz, a stimulation voltage of about 0.1 to 7.5 V, and a pulse width of about 0.4 to 3 msec. Moreover, continuous output of electrical pulses is effective for preventing arrhythmia, while intermittent output thereof is effective for improving engraftment of transplanted cells.

The intermittent output is achieved by, for example, repetition of one-minute stimulation pattern consisting of: a stimulation period with electrical pulses for 5 to 30 seconds; and a nonstimulation period without any electrical pulse for the rest of time.

Furthermore, the electrical pulses to be output are biphasic electrical pulses. By so doing, the tissue can be kept from being unipolarly charged.

Moreover, as shown in FIG. 7, the stimulating electrodes 3 are preferably arranged on a vagus nerve branch B1 or a vagus nerve ganglion B2. By so doing, the heart A can be exclusively stimulated while avoiding stimulation to other organs. The stimulating electrodes 3 may also be arranged on the root of the vagus nerve (vagus nerve trunk) B3.

The stimulating electrodes 3, if arranged on the vagus nerve branch B1, are preferably spaced 2 to 5 mm apart. By so doing, the area to be stimulated is not too much locally-limited, and the power for stimulation can be efficiently utilized to allow effective stimulation with saved power.

Moreover, regarding the stimulating electrodes 3 to be arranged on the vagus nerve B, the negative electrode (−) is preferably arranged closer to the heart A than the positive electrode (+). By so doing, excitation applied to the vagus nerve B by the negative electrode (−) can be readily transmitted to the heart A side. The neuronal excitation thus transmitted along the vagus nerve B towards the heart A side causes acetylcholine to be released from nerve endings, which leads to an advantage in that the viability of transplanted cells can be enhanced to achieve fast repair of the heart A.

In this manner, the regeneration treatment apparatus 1, the operating method thereof, and the regeneration treatment method according to the present embodiment are capable of improving engraftment of transplanted cells by stimulating the vagus nerve B without drug administration which involves side effects or risky gene transfection, and capable of increasing engraftment of such cells even as compared to drug administration.

That is to say, transplantation of mesenchymal stem cells in the heart A can increase engraftment of mesenchymal stem cells and enhance the therapeutic potency of mesenchymal stem cells, including improvement of cardiac systolic and diastolic function and prevention of ventricular remodeling in the heart A. In particular, stimulation to the vagus nerve B can provide beneficial effects through inhibition of apoptosis of mesenchymal stem cells, inhibition of inflammation and that of fibrosis of the heart A, and induction of angiogenesis. Furthermore, in the cell transplantation treatment for the heart A, arrhythmia occurs as a side effect of cell transplantation treatment per se. Stimulation to the vagus nerve B can also provide beneficial effects through inhibition of such occurrence of arrhythmia caused by cell transplantation treatment.

The present embodiment has been described by exemplifying the transplantation of mesenchymal stem cells into the heart A as an organ; however, the case is not limited thereto. The present invention is also applicable to other organs such as a liver C or a pancreas (not shown) which are greatly affected by the parasympathetic nerve. For example, treatments for hepatic cirrhosis through transplantation of stem cells can also be enumerated.

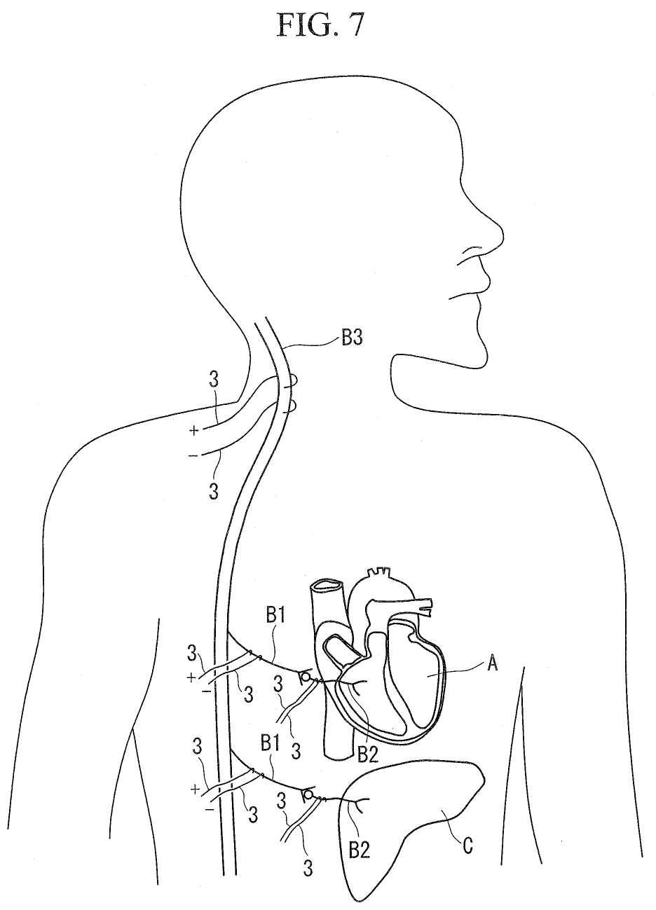

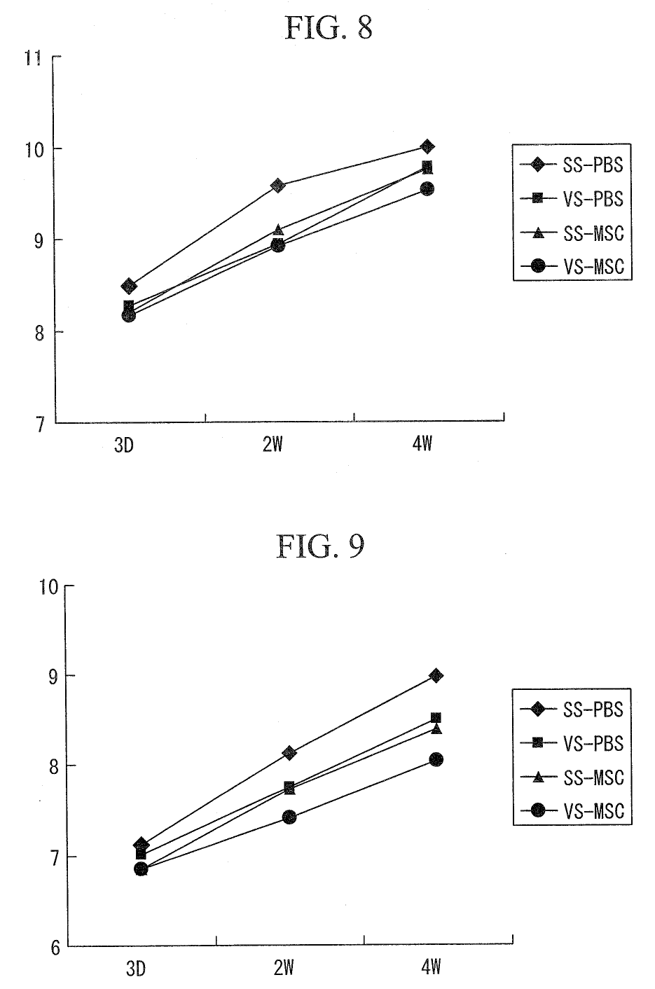

Next is a description of an example using rats to prove the effects of the regeneration treatment method with the regeneration treatment apparatus 1 according to the present embodiment, with reference to FIG. 8 to FIG. 18.

Bone marrow mesenchymal stem cells were transplanted for the treatment of rats with myocardial infarction. Then, heart rate levels corresponding to the electrocardiographic signal were calculated, and vagal stimulation was carried out. A conventional cell transplantation technique was used for comparison, and the comparison was made in the improving effect of cardiac function, the viability of transplanted cells, and the rate of angiogenesis. As a result, evident improving effects were observed in each of five sample animals.

### (Specific Methods)

At 1 hour after left coronary artery ligation, mesenchymal stem cells (5×10⁶ cells) expressing green fluorescent protein (GFP) were injected into the ischemic rat myocardium, followed by vagal stimulation (VS-MSC group) or sham stimulation (SS-MSC group) for 4 weeks.

A radio-controlled stimulator was implanted for the vagal stimulation. The stimulation intensity was adjusted for each rat to lower the heart rate by 20-30 beats/min. In another two groups of rats, phosphate buffered saline (PBS) was injected into the ischemic myocardium, followed by vagal stimulation (VS-PBS group) or sham stimulation (SS-PBS group).

Echocardiography was performed at 3 days, 2 weeks, and 4 weeks after myocardial infarction. At 4 weeks after myocardial infarction, cardiac catheterization was performed and transplanted hearts were harvested for histological analysis of transplanted cells and microvessel density.

### (Results)

Extensive myocardiac regeneration and survival of transplanted mesenchymal stem cells were observed in and around the infarcted myocardium in the VS-MSC group. Left ventricular diastolic dimension (LVDd) was smallest in the VS-MSC group, followed by the SS-MSC group and the VS-PBS group. LVDd was largest in the SS-PBS group (FIG. 8).

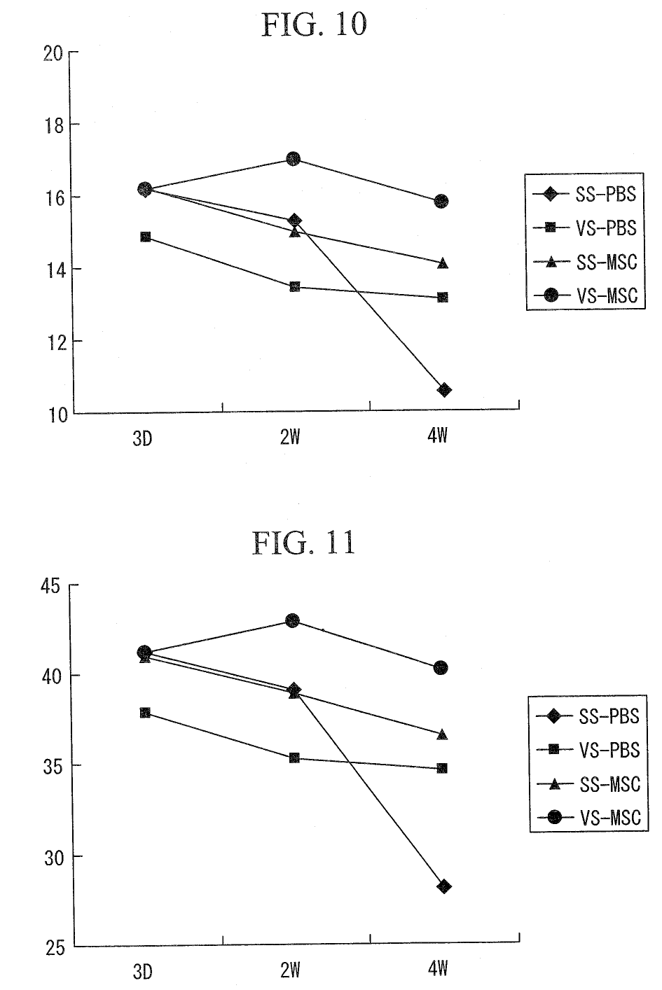

Left ventricular systolic dimension (LVDs) was smallest in the VS-MSC group, followed by the SS-MSC group and the VS-PBS group. LVDs was largest in the SS-PBS group (FIG. 9). Left ventricular fractional shortening (FS) was largest in the VS-MSC group, followed by the SS-MSC group and the VS-PBS group. FS was smallest in the SS-PBS group (FIG. 10).

Left ventricular ejection fraction (LVEF) was largest in the VS-MSC group, followed by the SS-MSC group and the VS-PBS group. LVEF was smallest in the SS-PBS group (FIG. 11).

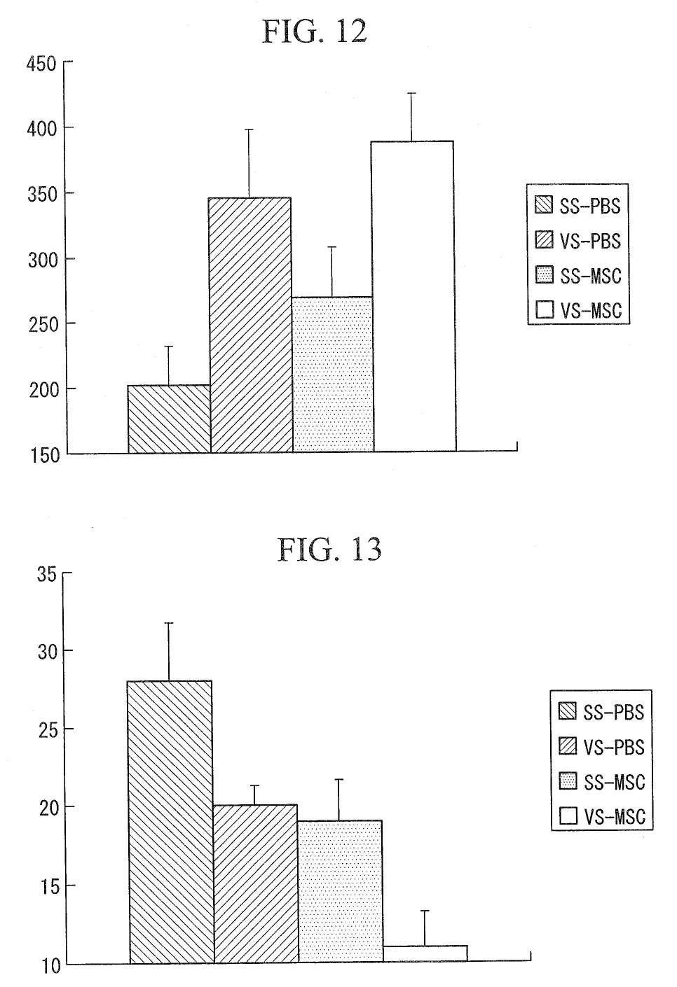

Left ventricular end-systolic elastance (Ees) was largest in the VS-MSC group, followed by the SS-MSC group and the VS-PBS group. Ees was smallest in the SS-PBS group (FIG. 12). Left ventricular end-diastolic pressure (LVEDP) was lowest in the VS-MSC group, followed by the SS-MSC group and the VS-PBS group. LVEDP was highest in the SS-PBS group (FIG. 13).

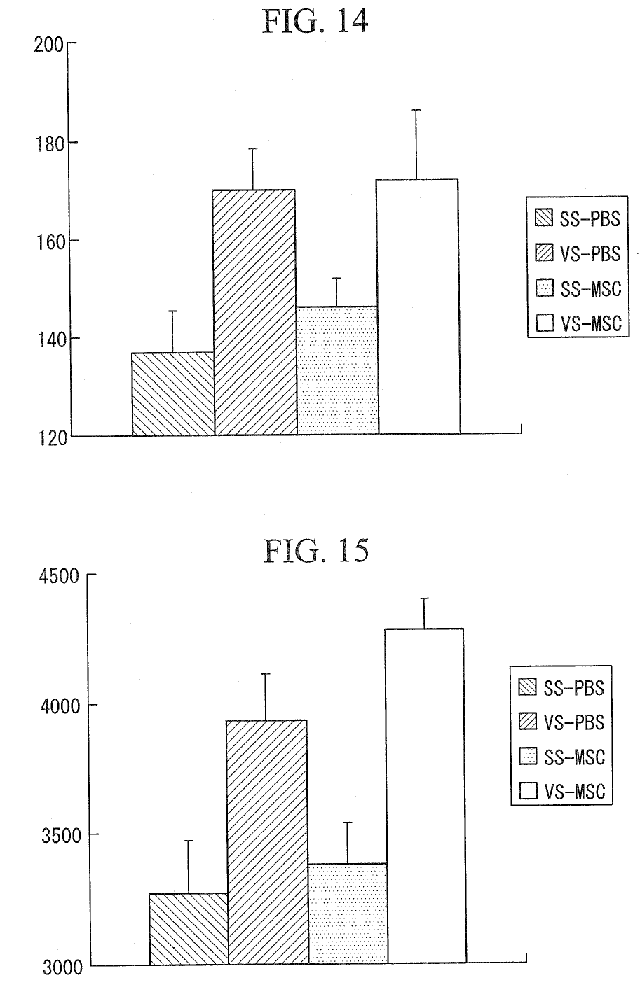

Cardiac index (CI) was largest in the VS-MSC group, followed by the SS-MSC group and the VS-PBS group. CI was smallest in the SS-PBS group (FIG. 14).

Maximum negative first derivative of left ventricular pressure (−dp/dtₘₐₓ) was largest in the VS-MSC group, followed by the SS-MSC group and the VS-PBS group. The −dp/dtₘₐₓ was smallest in the SS-PBS group (FIG. 15).

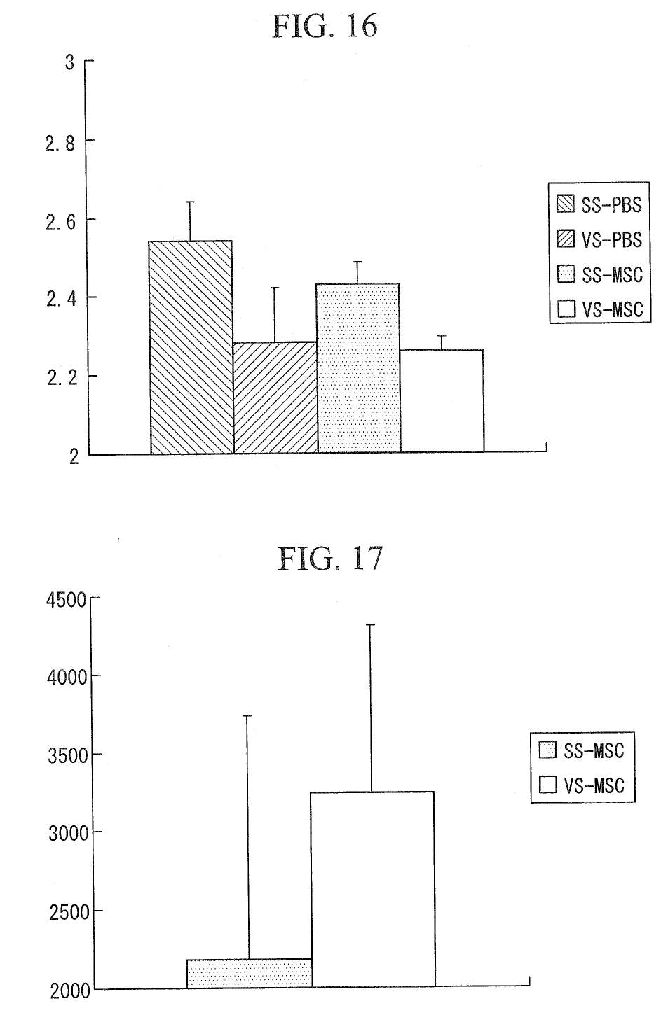

Ventricular weight normalized by body weight (VW) was smallest in the VS-MSC group, followed by the SS-MSC group and the VS-PBS group. VW was largest in the SS-PBS group (FIG. 16).

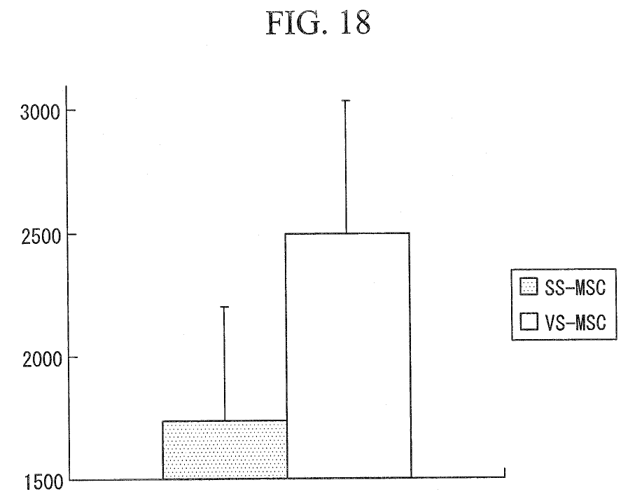

Semiquantitative evaluation in the histological analysis demonstrated that the number of viable transplanted cells was higher in the VS-MSC group than in the SS-MSC group (FIG. 17). Microvessel density was higher in the VS-MSC group than in the SS-MSC group (FIG. 18).

### (Conclusion)

Vagal stimulation increased engraftment of transplanted mesenchymal stem cells and enhanced the therapeutic potency of mesenchymal stem cells, including improvement of cardiac systolic and diastolic function and prevention of ventricular remodeling. These beneficial effects of vagal stimulation may be mediated partly by the inhibition of apoptosis of mesenchymal stem cells and induction of angiogenesis.

1. A regeneration treatment apparatus comprising:
a heart rate detector which detects a heart rate of a patient;
a memory which stores a heart rate prior to stimulation;
stimulating electrodes which stimulate a vagus nerve that controls an organ having transplanted cells; and
a control unit which controls an intensity of a stimulation signal to be output from the stimulating electrodes to the vagus nerve so that the heart rate of the patient detected by the heart rate detector is decreased by 5 to 20% as compared to the state prior to the stimulation.

2. A regeneration treatment apparatus according to claim 1, wherein the stimulation signal takes a pulse-like form having a frequency of about 10 Hz, a stimulation voltage of about 0.1 to 7.5 V, and a pulse width of about 0.4 to 3 msec.

3. A regeneration treatment apparatus according to claim 1, wherein the stimulation signal is either intermittently or continuously output from the stimulating electrodes.

4. A regeneration treatment apparatus according to claim 1, wherein the stimulation signal is intermittently output from the stimulating electrodes by repetition of one-minute stimulation pattern consisting of: a continuous stimulation period for 5 to 30 seconds from the stimulating electrodes; and a nonstimulation period for the rest of time.

5. A regeneration treatment apparatus according to any claim 1, wherein the stimulation signal is an electrical stimulation signal consisting of biphasic pulses.

6. An operating method of a regeneration treatment apparatus, comprising:
operating a heart rate detector which detects a heart rate of a patient;
operating a memory which stores a heart rate prior to stimulation;
operating a stimulation signal generator which generates a stimulation signal to stimulate a vagus nerve;
operating a determination unit which determines whether or not the heart rate detected by the heart rate detector is decreased by 5 to 20% as compared to the heart rate stored in the memory; and
operating a control unit which increases the intensity of the stimulation signal generated from the stimulation signal generator if the determination unit determines that the reduction of the heart rate is less than 5%, and lowers the intensity of the stimulation signal if the determination unit determines that the reduction of the heart rate exceeds 20%.

7. An operating method of a regeneration treatment apparatus according to claim 6, wherein the stimulation signal takes a pulse-like form having a frequency of about 10 Hz, a stimulation voltage of about 0.1 to 7.5 V, and a pulse width of about 0.4 to 3 msec.

8. An operating method of a regeneration treatment apparatus according to claim 6, wherein the stimulation signal is either intermittent or continuous.

9. An operating method of a regeneration treatment apparatus according to claim 6, wherein the stimulation signal is an intermittent stimulation signal achieved by repetition of one-minute stimulation pattern consisting of: a continuous stimulation period for 5 to 30 seconds; and a nonstimulation period for the rest of time.

10. An operating method of a regeneration treatment apparatus according to claim 6, wherein the stimulation signal is an electrical stimulation signal consisting of biphasic pulses.

11. A regeneration treatment method, comprising: transplanting cells into an organ; and stimulating a vagus nerve that controls the organ having the transplanted cells.

12. A regeneration treatment method according to claim 11, wherein the stimulation site of the vagus nerve is a vagus nerve trunk, a vagus nerve branch, or a vagus nerve ganglion.

13. A regeneration treatment method according to claim 11, wherein the heart rate of the patient is detected and the intensity of stimulation to the vagus nerve is adjusted so that the detected heart rate is set to a predetermined heart rate.

14. A regeneration treatment method according to claim 13, wherein the stimulation intensity is adjusted so that the detected heart rate is decreased by 5 to 20% as compared to the state prior to the stimulation.

15. A regeneration treatment method according to claim 11, wherein the stimulation to the vagus nerve is a pulse-like stimulation having a frequency of about 10 Hz, a stimulation voltage of about 0.1 to 7.5 V, and a pulse width of about 0.4 to 3 msec.

16. A regeneration treatment method according to claim 15, wherein the pulse-like stimulation is either intermittently or continuously performed.

17. A regeneration treatment method according to claim 15, wherein the pulse-like stimulation is intermittently performed by repetition of one-minute stimulation pattern consisting of: a continuous stimulation period for 5 to 30 seconds; and a nonstimulation period for the rest of time.

18. A regeneration treatment method according to claim 15, wherein the pulse-like stimulation is an electrical stimulation performed by biphasic pulses.

19. A regeneration treatment method according to claim 11, wherein a positive electrode and a negative electrode for stimulation are spaced 2 to 5 mm apart to be arranged on the vagus nerve.

20. A regeneration treatment method according to claim 19, wherein the negative electrode is arranged closer to the organ than the positive electrode.
